(19) United States
(12) Patent Application Publication
(10) Pub. No.: US 2004/0180005 A1
(43) Pub. Date: Sep. 16, 2004

(54) Method for the production of nanodispersions

(76) Inventors: Kai Christian Jurgens, Berlin (DE); Bernd Kuhn, Koln (DE)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/478,601

(22) PCT Filed: May 8, 2002
(86) PCT No.: PCT/EP02/05048

(30) Foreign Application Priority Data
May 21, 2001 (DE) 101 24 952.7

(51) Int. Cl. A61L 9/04; A61K 9/14

(52) U.S. Cl. 424/46

(57) ABSTRACT

A method for the production of nanodispersions, in which at least two metered partial streams are brought together in such a way that they are subject to thorough mixing caused by turbulence. The partial streams have a flow rate in the range from 0.1 to 500 ml/h and the mixed stream has an overall flow rate in the range from 1 ml/h to 500 ml/h. The turbulent mixing leads to the formation of a disperse phase in a continuous phase with a dispersity of 0.1 to 5000 nm. Another object of the invention is a method for the in-situ formulation of a pharmaceutical dispersion, with in-line application of the pharmaceutical dispersion.

The invention relates to the production of nanodispersions especially for the application of pharmaceuticals in humans and animals and a method for the in-situ formulation of a pharmaceutical dispersion. The invention relates in particular to a method for the production of phospholipid adducts, which can be used as pharmaceutical formulations.

Application, according to the invention, of the method described is not restricted to the production of pharmaceutical end products, but is preferably used for these.

In the sense of this invention, the term “nanodispersion” describes a disperse system with a disperse phase, which can be liquid, liquid-crystal, gaseous, vesicular or micellar and can be of organic or inorganic origin, in a dispersion medium (the continuous phase), which can be composed of one or more components.

For characterization of nanodispersions, in the scope of this invention we determine the dispersity, by measuring the dispersion using dynamic light scattering (photon-correlation spectroscopy). The disperse phase is regarded as being made up of solid, spherical particles, so that the dispersity can be stated as the mean hydrodynamic diameter of these virtual particles.

The characteristic of the nanodispersion according to the invention is that the dispersity is in the range 1-5000 nm, the size distribution being such that the amount of the disperse phase larger than 1000 nm is small in relation to the total amount. If the disperse phase consists of a solid, we have—as a special form of nanodispersion—a nanosuspension, in which dynamic light scattering measures solid particles.

“In-situ formulation” means that final formulation of the pharmaceutical takes place just before application.

In the following, “turbulent mixing” will be understood as meaning that at least two partial streams move in such a way that their flow lines follow chaotic paths, so that it can be assumed that the phases that are formed from the partial streams are distributed statistically uniformly in the available space. The term is used without the connotations of the definition of turbulence in flow mechanics.

In the following, the term “a pharmaceutical” means a substance that leads to a medicinal product according to the German Drug Law (AMG) § 2 Paragraph 1 and Paragraph 2 when used appropriately, or is defined as a substance in the sense of the German Drug Law (AMG) § 2.

When the term “substance” is used in the following, this means chemical substances, preferably pharmaceuticals, though not restricted to the latter.

“Parenteral application” means in particular an intravenous, intra-arterial, intramuscular, subcutaneous, intraperitoneal, intrathecal or intracardiac injection or infusion.

For the action of a pharmaceutical in the patient's body, molecular solution of the pharmaceutical is a precondition in the vast majority of cases. However, a large number of modern pharmaceuticals have very poor solubility in media that are compatible for the human body. Pharmaceutical technology is therefore faced with the challenge of finding formulation solutions for this constantly growing group of sparingly soluble pharmaceuticals. “Sparingly soluble” means that at least 100 parts water are required for dissolving 1 part of the substance.

It is especially difficult to find formulations for parenteral applications, as the choice of excipients and methods is very limited here. Furthermore, the pharmacopeias state requirements on the number and size of particulate constituents in a medium for parenteral application. For example, the US Pharmacopeia stipulates that large-volume infusion solutions may not contain more than 25 particles larger than 10 μm per mL. This is based on the size of the erythrocytes, which with a diameter of approx. 5-7 μm can pass through all the body's capillaries. If individual constituents are larger, there is a danger that they will be retained in the body's capillaries, block them and thus cause injuries to the body.

Although these regulations are intended to regulate the contamination of true solutions with xenoparticles, they are also binding for nanodispersions.

In difficult operations, often the patient's blood is enriched with oxygen externally with oxygenators, in the scope of extracorporeal circulation. The small size of the gas bubbles is decisive for rapid physical uptake of the gas into the blood.

In this process, the gases used behave as very hydrophobic substances; they do not dissolve in the blood and can, moreover, coalesce to form larger bubbles.

In some cases it is therapeutically desirable to bring gas bubbles directly into the bloodstream, e.g. for X-ray contrast purposes. The formation of large gas bubbles can lead in this case to an embolism, the blocking of a blood vessel.

One solution is for the sparingly soluble pharmaceuticals, or gases, to be applied in undissolved form, but in such a fine dispersion that they can pass through all the capillaries without any problem. There are numerous proposals for methods of production for formulations of this kind.

An important precondition for successful formulation of such dispersions is stabilization of the insoluble constituents at a size that can pass through capillaries. This stabilization must remain effective for the entire shelf life of the pharmaceutical.

In U.S. Pat. No. 5,145,684, G. G. Liversidge et al. use wet grinding, for reducing the active substance to the required particle size <5 μm. To prevent agglomeration and particle growth, surfactants such as polyvinylpyrrolidone or poloxamers are added to the grinding medium.

Lucks et al. produce, according to EP 0 605 497, solid lipid nanospheres (SLN) containing the active substance, by melting one or more lipids, incorporating the active substance, mixing with water and comminuting using high shearing forces (Ultra-Turrax and high-pressure homogenizer). Stabilizers can be added for stabilizing the formulation thus obtained.

List et al. disclose, in DE 3742473, a method in which ciclosporin is dissolved in an organic solvent and is then introduced into an aqueous solution of a stabilizer. The purpose of the stabilizer, preferably gelatin or ethylcellulose, is to stabilize the extreme degree of dispersion produced by the solution, by coating the surface of the particles that form.

In WO 92/18105, Gassmann et al. extend this method, in that, firstly, they dissolve a water-insoluble active substance in an organic solvent, with addition of a charged phospholipid, and then mix it with an aqueous solution, which can contain further stabilizers. The polarity of the solvent mixture is altered to such an extent that the solubility of the active substance is exceeded and it is precipitated. The charged phospholipid coats the surface of the particles that form and stabilizes them. The authors describe various methods of mixing, including continuous mixing with a static mixer.

The formation of very small vesicles, with lamellar formation of their surface from phospholipids, is common knowledge. A preferred method for their production is described by Rahman et al. in U.S. Pat. No. 5,648,090: the active substance is dissolved together with phospholipid and possibly with other excipients in an organic medium, which is then removed again by means of a rotary evaporator. Water, which may contain other excipients, such as stabilizers or additives for increasing isotonicity, is added to the thin film that has formed.

An aqueous dispersion forms from multilamellar phospholipid vesicles (MLV). This is then converted, using ultrasound, into a dispersion of small unilamellar vesicles (SUV). These vesicles are also called liposomes.

Comminution of MLV to SUV by high-pressure homogenization is also known from the literature.

In EP 0 616 801, Frederiksen et al. disclose a method for producing liposomes using supercritical gases.

In EP 0 956 851, Hüiglin et al. disclose the possibility of forming lipid-containing dispersions with vesicles in the submicron range even without further energy input. For this they employ special formulations, which are characterized by the addition of a co-emulsifier (e.g. Tween® or Pluronic®).

In WO 97/30695, for production of a microemulsion, Yiv et al. use a concentrate in which they dissolve an active substance in a mixture of a phospholipid with propyleneglycol or polyethyleneglycol. They relied on the use of a surfactant with HLB value of >12. This concentrate is then mixed with water shortly before use.

With this manner of production it is possible to overcome the problem of long-term storage and therefore stabilization of the ready-to-use dispersion. However, because the ready-to-use solution only remains usable for a short time, interruption of application or very slow administration is only possible in certain circumstances. Residual amounts of the formulations, which are often very expensive on account of the active substance, have to be destroyed.

An in-situ formulation, i.e. a formulation that only forms at the time of application, is described by Leigh in WO 99/29301. He dissolves an active substance with a phospholipid in ethanol and glycerol. If this formulation is applied to the mucosa, lysosomes or similar structures, containing the active substance as a molecular dispersion, are formed spontaneously by the liquid that is present there. He describes a fungicidal dosage form as examples. In EP 0 759 736 he describes a similar formulation for production of bath oils.

In WO 99/44642, Leigh extends the pre-liposomal concept to non-topical dosage forms as well. He dissolves phospholipids in a water-miscible solvent and adds the active substance. If this premixture is now hydrated, phospholipid aggregates form, which are also said to contain bilayer structures. As these structures are formed spontaneously without further energy input, it is necessary to use quite special mono- and diacylated phospholipids, which are produced by enzymatic cleavage.

Preferably the formulations are used orally. In that case the phospholipid aggregates form on contact with the gastric juice. They contain the active substance as a molecular dispersion.

What the above concepts have in common is that they require additional substances for stabilizing the particulate application dispersions, which can lead to unwanted reactions in and on the patient, which impose a strain on the patients in addition to the active substance. The in-situ formulations described do not possess any possibility of controlling and possibly influencing the dispersity of the dispersions produced. Therefore they are only suitable for parenteral application with certain provisos.

The problem of the invention is therefore to find a method for the production of nanodispersions with good tolerability, which is suitable for the in-situ formulation of sparingly soluble pharmaceuticals with application following immediately. The application form can be parenteral, oral or topical.

The solution of the problem according to the invention consists of a method for the production of nanodispersions in which at least two metered partial streams are brought together in such a way that they undergo mixing caused by turbulence, characterized in that the partial streams have a flow rate in the range from 0.1 to 500 ml/h and the mixed stream has an overall flow rate in the range from 1 ml/h to 500 ml/h, preferably in the range from 10 to 200 ml/h, and in that, during the turbulent mixing, a disperse phase is produced with a dispersity in the range from 0.1 to 5000 nm, preferably in the range from 10 to 1000 nm, and, especially preferred, in the range from 10 to 200 nm.

With suitable geometry of the mixing device and parameters of the partial streams, turbulent mixing of the two or more partial streams is achieved in that the partial streams flow through a nozzle into a discharge channel, the nozzle having a smaller diameter than the discharge channel. The mixed stream is produced by bringing the two partial streams together. The sum of the flow rates of the partial streams gives the overall flow rate.

The following method can be employed for selecting the geometric relations and the parameters of the partial streams to give turbulent mixing of the partial streams:

$$K = \frac{{r}_{channel} \cdot ρ \cdot \dot{v}}{η \cdot {r}_{nozzle}^{2} \cdot π} \qquad Eq . 1$$

A characteristic number K can be calculated from Eq. 1. In the above equation r_{channel} is the radius of the discharge channel, ρ is the density of the mixture, \dot{v} is the overall flow rate, η is the viscosity of the mixture, r_{nozzle} is the radius of the nozzle and π is the ratio of the circumference of a circle to its diameter. For the calculation, all values are to be used in the corresponding SI units.

If this characteristic number exceeds a critical value, mixing will be turbulent. The critical value is in the range from 250 to 450. As well as the aforementioned parameters, it depends to a lesser extent on the precise nozzle geometry, the surface condition of the walls, the temperature and the interfacial tension between the partial streams used.

If, for given geometric relations, we employ an overall flow rate that leads to a characteristic number K which is below the critical range, flow through the mixer will be laminar. The partial streams enter the nozzle next to one another. There they are accelerated and enter the discharge channel with a corresponding velocity. There is a sudden drop in pressure owing to the expansion of the channel diameter. The velocity of the partial streams is still low enough so that they can leave the original direction, following the pressure gradient, and spread out in the whole discharge channel. Laminar flow is maintained, and no mixing occurs.

At a higher overall flow rate the energy of the partial streams, on discharge from the nozzle into the discharge channel, is so high that they can no longer adjust their direction of motion to the enlarged space. A jetstream forms, traveling centrally through the discharge channel and then expanding to the full width of the channel. Before the stream reaches the channel wall, molecules of liquid from the immediate vicinity adhere to its surface and are entrained. Directly behind the nozzle, a region forms around the stream with a relative deficit, which is compensated from the surroundings, so that a zone of negative pressure forms, which can be compensated in its turn when, at some distance from the nozzle, material leaves the stream and fills the negative-pressure region. Owing to the distance from the nozzle, the velocity of the material leaving the stream is reduced to such an extent that the force of suction is greater than the kinetic energy of the particles. Accordingly, an eddy forms concentrically around the nozzle and surrounds the jetstream like a ruff. There, liquid from remoter regions is brought back to the nozzle. The rotational speed of the eddy depends on the velocity of the jetstream. There is a state of turbulence just behind the nozzle. Starting from the point where the jetstream meets the channel wall, the flow of the stream is laminar again.

The turbulent eddy zone ensures thorough mixing of the partial streams. However, the zone must not extend too far, as otherwise exchange of material within the eddy will not take place quickly enough, with the result that the eddy reduces the dispersity.

Formation of the turbulent eddy zone ensures optimum mixing of the two partial streams. A further increase in flow rates increases the throughput, but does not improve mixing. Turbulent mixing is achieved at an overall flow rate that is above a critical overall flow rate at which turbulence sets in. This critical overall flow rate depends on the ratio of the diameters of the nozzle and the discharge channel, the geometry of the nozzle and of the discharge channel, and on the material properties of viscosity and density of the partial streams and of the mixed stream.

Preferably the discharge channel has a diameter between 0.2 and 2 mm and the nozzle a diameter in the range from 10 to 500 μm. The length of the discharge channel is preferably at least 10 times longer than its diameter. The mixed stream preferably has a viscosity in the range 0.7 mPas to 150 mPas and the density is between 700 kg/m³ and 1500 kg/m³. The parameters overall flow rate, diameters of the nozzle and discharge channel, viscosity and density are related to one another in such a way that Eq. 1 gives a characteristic number K of at least 250.

It was found that with identical mixing conditions and given geometric relations, the dispersity of the disperse phase depends on the overall flow rate employed. With a low overall flow rate, at first we get a low dispersity. On increasing the overall flow rate, mixing becomes more uniform, and the dispersity increases until it reaches a maximum value, beyond which no further significant increase is possible even if there is a further increase in overall flow rate.

In addition, it was established that the range of overall flow rate that leads to a high dispersity coincides with the range of overall flow rate at which turbulent mixing develops behind the nozzle.

Turbulent mixing of the partial streams is therefore an essential requirement for the production of nanodispersions with a high dispersity. It was found, moreover, that the shelf life of a nanodispersion with corresponding high dispersity is greater, relative to a dispersion with a lower dispersity.

To increase the overall throughput, parallel connection of several mixers is also possible. Furthermore, several mixers can also be connected one after another for the production of premixtures of various components.

To produce the formulation, at least two partial streams are mixed using the aforementioned method of mixing, so that a nanodispersion is produced.

These nanodispersions consist of a continuous phase and a disperse phase, with a very high dispersity.

The disperse phase can be a solid, a liquid, a liquid-crystal phase, a gas or a mixture thereof.

Reasons for formation of the disperse phase are precipitation on account of the saturation solubility of the solution being exceeded, neutralization reaction, interaction between differently charged molecules, association of molecules, recomplexing or chemical reaction. Which of the causes applies depends on the choice of materials or mixtures of materials in the partial streams.

The continuous phase can be water or distilled water or an aqueous medium or an aqueous medium with additions of electrolytes, monosaccharides or disaccharides, alcohols, polyols or their mixtures.

The continuous phase can contain one or more viscosity-raising substances.

The continuous phase can contain stabilizers and/or surfactants.

Preferably the continuous phase is water for injection, without addition of stabilizers or surfactants, though adjuvants for adjusting isotonicity and euhydration can be added. In an especially preferred embodiment, 5% glucose is added to the water to make it isotonic.

In another preferred embodiment the continuous phase contains additives that form micelles in the application conditions. After mixing, the disperse phase is inside these micelles. Additives that fulfill these conditions are for example substances from the poloxamer series. Poloxamer 408 is especially preferred.

On the basis of in-situ production, the disperse phase only forms when the partial streams are mixed, immediately before application. At least one of the partial streams therefore contains the later disperse phase or parts of the later disperse phase in dissolved form. It is also possible that the partial stream in its entirety constitutes the later disperse phase, for example with direct dispersion of gases.

In an especially preferred embodiment of the invention, the disperse phase is produced by a partial stream which will be called concentrate hereinafter.

The concentrate consists of an aqueous or water-miscible organic solvent, which preferably is permitted for parenteral use.

Solvents that are especially preferred are water, polyethyleneglycol 400, propyleneglycol, ethanol, tetraglycol and glycofurol.

According to the invention, other excipients can be added to the concentrate, which are known to a person skilled in the art in type and quantity and are required for example, though not exclusively, for pH adjustment, preservation, complexing, raising or lowering the viscosity or for attainment of chemical stability.

Substances that are sparingly soluble or practically insoluble in water can be added to the concentrate in a sufficient quantity for pharmaceutical purposes e.g. one or more pharmaceuticals. These substances can be dissolved in the concentrate.

The concentration of the active substances can be between 0 and 50 wt. %, preferably between 0.1 and 10%. In a quite especially preferred embodiment the concentration is between 1% and 3%. The active substance is preferably a pharmaceutical from the following group: cardiovascular agents, oncologic agents, virostatic agents, analgesics, chemotherapeutics, hepatitis agents, antibiotics or immunomodulators. The active substance can also be a gas, e.g. NO for vasodilatation, O₂ for oxygenation or air as X-ray contrast agent.

The anhydrous embodiment of the concentrate can, with some added pharmaceuticals, lead to improved chemical stability compared with an aqueous solution or suspension.

However, it is also possible for the concentrate to contain water.

The method according to the invention is especially suitable for the production of phospholipid adducts as disperse phase, which can be used as pharmaceutical formulations. It was found that when phospholipids dissolved in water-miscible organic media (first partial stream) are mixed with water (second partial stream) without further additions, using the method according to the invention, phospholipid adducts form that have a dispersity in the nanometer range and can be used as pharmaceutical formulations.

In a preferred embodiment, the concentrate therefore contains a phospholipid or a mixture of several phospholipids dissolved in a water-miscible organic solvent. This can consist of an anhydrous mixture of 10 to 50, preferably 25 to 35 parts by weight ethanol with 50 to 90, preferably 65-75 parts by weight polyethyleneglycol 400 (PEG 400).

The concentrate preferably contains a phospholipid, a hydrogenated or partially hydrogenated phospholipid, a lysophospholipid, a ceramide or mixtures of these compounds. Phospholipids with the trivial names lecithin or kephalin are especially preferred; the following are quite especially preferred: purified lecithins from soybeans of the grades Epikuron 170, Epikuron 175, Lipoid S100 or S75 and purified lecithins from egg yolk of the grades Lipoid E80, E100 and EPC. The percentage by weight of the phospholipid in the concentrate can be between 0.01% and 40%, preferably between 5% and 20%. In an especially preferred embodiment it is 9-11%.

In the especially preferred embodiment, the mixer according to FIG. 3 already known from patent WO 99/32175 is used for mixing the partial streams, and is constructed so that the nozzles have a cross-section of approx. 100 μm.

In the especially preferred application, the concentrate and the diluting solution are each supplied to the mixer via a jet pump (e.g. Perfusor® Compact from the company B. Braun, Melsungen). The flow rate of the partial streams is selected so that the flow rate of the dispersing medium is 8-11 times, preferably 9 times, higher than that of the concentrate and an overall flow rate between 80 and 110 ml/h is obtained.

If the two partial streams are brought together in accordance with the method of the invention, a mixture is produced that contains phospholipid adducts, with which an active substance is associated in such a way that precipitation of the active substance does not occur, which would take place without addition of the phospholipid.

The phospholipid adducts produced according to the invention have a dispersity between 10 and 1000 μm. The quantity of adducts that are larger than 2000 nm is then very small relative to the total quantity. In the preferred embodiment the dispersity is between 10 and 500 nm.

Another preferred embodiment comprises a nanodispersion in which the disperse phase is formed from a gas. In this case one of the partial streams can be a gas, which can be dispersed by the mixing process as extremely fine bubbles in the diluent and can be stabilized by the latter against coalescence.

In another embodiment of the invention, the gas is first produced by a chemical reaction involving reactants that are dissolved in different partial streams.

For example, one stream can contain NaHCO₃, whereas the other stream contains an acid. When they are mixed, carbon dioxide forms as gas, which has a very high dispersity.

In a further preferred embodiment, the nanodispersion is produced for example by a neutralization reaction, in which a pharmaceutical is dissolved in an aqueous solvent at a non-physiological pH and is mixed with a neutralizing diluent in the mixer. At the resulting pH the substance is only sparingly soluble and is precipitated as a disperse phase in the continuous phase.

The mixing ratio of the concentrate and of the diluting solution can be fixed or temporarily variable. According to the invention, it is arranged so that the proportion by volume of concentrate in the total mixture is between 0 and 90%, preferably between 1 and 50%. The embodiment that is especially preferred uses a fixed mixing ratio of 1 part concentrate in 10 parts mixture.

A further object of the invention is a method for in-situ formulation of a pharmaceutical dispersion, the pharmaceutical dispersion being produced at the same rate at which application takes place, so that the total quantity produced can be applied immediately (in-line application). In this case the pharmaceutical dispersion is produced by a method in which at least two metered partial streams are brought together in such a way that they are subject to thorough mixing caused by turbulence, with at least one partial stream containing a pharmaceutical and with the partial streams having a flow rate in the range from 0.1 to 500 ml/h and the mixed stream having an overall flow rate in the range from 1 ml/h to 500 ml/h, preferably in the range from 10 to 200 ml/h, and with the turbulent mixing leading to production of a disperse phase with a dispersity in the range from 0.1 to 5000 nm, preferably in the range from 10 to 1000 nm, and—especially preferred—in the range from 10 to 200 nm.

Preferably the pharmaceutical dispersion is applied parenterally. This method including the parenteral in-line application of the pharmaceutical dispersion can be carried out without risk for the patient, because as a result of the turbulent mixing, the disperse phase produced has a dispersity that is below the critical particle size for parenteral application, and at the same time the overall flow rate is in a range of up to 500 ml/h.

The principal application of the method for the production of nanodispersions is the in-situ formulation of pharmaceutical dispersions for parenteral application in humans and animals. Other possible uses are oral, ophthalmologic, otologic, topical, nasal, vaginal, urethral and rectal application in humans and animals. Production of the pharmaceutical formulations by the method according to the invention can of course also be carried out in such a way that the dispersion produced is not applied directly. In this case it is possible to add excipients as well, if required, to the dispersion for stabilization.

Preferably the mixture is applied parenterally, and especially intravenously. Application of the mixture can, however, also be oral, ophthalmic, topical, nasal, vaginal, urethral or rectal. It may be necessary to add other excipients not yet mentioned, which are known in type and quantity to a person skilled in the art. According to the invention, the mixture can be applied directly or with a time delay, direct application being preferred.

In another form of application it is possible to add one or more active substances to the diluting solution.

The nanodispersions produced by the method according to the invention possess a dispersity in the nanometer range and are therefore also available for parenteral application. Any active substance that is present can be dissolved in the disperse phase as a molecular dispersion.

An advantage of the formulations produced according to the invention is that they only contain components that are safe in particular for parenteral use. The formulation is characterized by very high compatibility. It therefore differs from other formulations known in the literature, which contain excipients with only limited compatibility, for example some ionic or non-ionic emulsifiers. If formulation is effected in situ according to the method of the invention, it may be unnecessary to add stabilizers.

The embodiment of the method for production of nanodispersions permits the use of generally available, commercial phospholipids, poloxamers or other surfactants.

### DIAGRAMS AND EXAMPLES

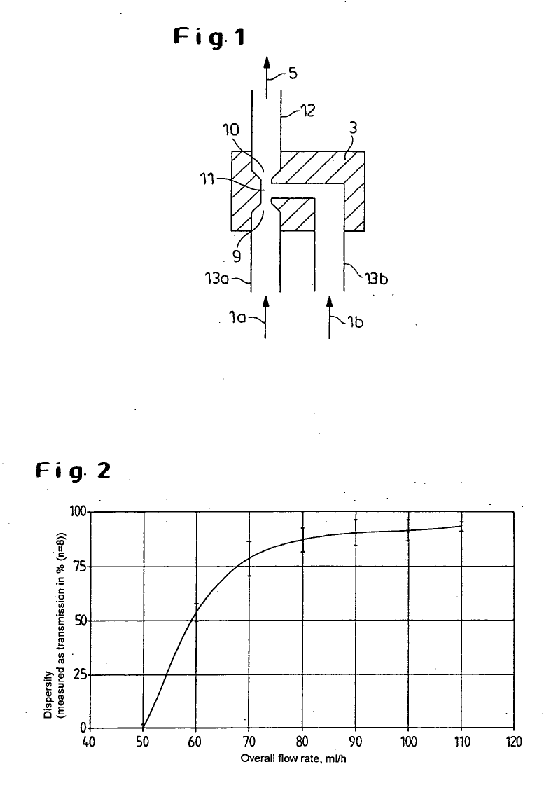

FIG. 1 Preferred embodiment of a mixer

FIG. 2 Graph of dispersity as a function of overall flow rate

FIG. 1 shows a preferred embodiment of a static mixer for carrying out the method according to the invention. The mixer is known from WO 99/32175, FIG. 3. Mixer 3 comprises a housing with a first channel 13a and a second channel 13b, a nozzle region 9, 10, 11 and a discharge channel 12. The first channel 13a and the second channel 13b serve as feed lines for the partial streams 1a and 1b. In the preferred application, the organic, water-miscible concentrate containing the phospholipid is supplied to the mixer as partial stream 1b via feed channel 13b. The continuous phase enters the mixer as partial stream 1a via channel 13a and is accelerated by the constriction of channel 9 into the nozzle 11. A metered amount of the concentrate is added here. After nozzle 11, in zone 10 the two streams 1a and 1b are mixed intimately by intensive longitudinal mixing, and they then leave the mixer as mixed stream 5 via the following discharge channel 12. Discharge channel 12 is arranged in the extension of the first feed channel 13a and at an angle of 90° to the second feed channel 13b.

Determination of Dispersity as a Function of Flow Rate

FIG. 2 shows a graph of the dispersity as a function of the overall flow rate.

It is known from Mie's equations that the intensity of scattered light increases as the dispersity decreases. Therefore the dispersity can be determined from measurements of turbidity. Measurement of turbidity is an additional method, alongside measurement in the photon correlation spectroscope, for determining dispersity. If turbidity is found as reciprocal transmission in a spectrophotometer, the dispersity correlates directly with the transmission.

For the determination of dispersity as a function of the flow rate, the placebo-concentrate described in example 1 was used as the concentrate. Water was used as the continuous phase. The dispersity was determined by measuring the transmission, with which it is correlated directly, on a UV-VIS photometer (Lambda 2, Perkin Elmer) at 620 nm. It had been confirmed in a preliminary test that the mixture does not possess any notable absorption at 620 nm.

The concentrate and the continuous phase were each drawn up into a 50 ml Perfusor® syringe and each was inserted in a Perfusor®-Compact syringe pump. The syringes were connected to a mixer according to FIG. 1 with nozzle cross-sections of 100 μm by hoses and the inlets of the mixer were provided with non-return valves.

With a fixed mixing ratio of 1 part concentrate plus 9 parts continuous phase, the flow rate was raised from 50 ml/h (5 ml/h concentrate, 45 ml/h continuous phase) to 110 ml/h (11 ml/h concentrate, 99 ml/h continuous phase). The turbidity of the mixture steadily decreased and accordingly the transmission and the dispersity steadily increased.

It can be seen from FIG. 2 that the dispersity depends on the flow rate. The dispersity is low at low flow rates and then increases as the flow rate increases. Starting from a break point at about 80 ml/h, there is no longer any marked change in dispersity even with further increase in flow rate. At this flow rate, it was possible, using investigations by microscopy, to observe the start of turbulent flow in the mixer.

### Example 1

### Concentrate Without Active Substance

| | |
|---|---|
| Epikuron 170 | 1000 mg |
| Ethanol | 2700 mg |
| PEG 400 | 6300 mg |

Production: Mix together ethanol and PEG 400. Add Epikuron 170 and dissolve, with stirring. Filter the solution in sterile conditions through a nylon filter. The solution is clear, particle-free, and of a yellow color.

### Example 2

### Nimodipine Concentrate 1%

| | |
|---|---|
| Nimodipine | 100 mg |
| Epikuron 170 | 1000 mg |
| Ethanol | 2700 mg |
| PEG 400 | 6200 mg |

Production: Mix together ethanol and PEG 400. Add nimodipine and Epikuron 170 and dissolve, with stirring. Filter the solution in sterile conditions through a nylon filter. The solution is clear, particle-free, and of a dark-yellow color.

### Example 3

### Ibuprofen Concentrate 1%

| | |
|---|---|
| Ibuprofen | 100 mg |
| Epikuron 170 | 1000 mg |
| Ethanol | 2700 mg |
| PEG 400 | 6200 mg |

Production: as in Example 2. The solution is clear, particle-free, and of a yellow color.

### Example 4

### Clotrimazole Concentrate 0.5%

| | |
|---|---|
| Clotrimazole | 50 mg |
| Epikuron 170 | 1000 mg |
| Ethanol | 2700 mg |
| PEG 400 | 6250 mg |

Production: as in Example 2. The solution is clear, particle-free, and of a yellow color.

### Example 5

### Paclitaxel Concentrate 1%

| | |
|---|---|
| Paclitaxel | 100 mg |
| Epikuron 170 | 1000 mg |
| Ethanol | 2700 mg |
| PEG 400 | 6200 mg |

Production: as in Example 2. The solution is clear, particle-free, and of a yellow color.

### Example 6

### Concentrate 1% of a Taxoid Active Substance

| | |
|---|---|
| Taxoid active substance | 100 mg |
| Epikuron 170 | 1000 mg |
| Acid. Sorbic. | 50 mg |
| Ethanol | 2700 mg |
| PEG 400 | 6200 mg |

Production: Mix together ethanol and PEG 400. Add the taxoid active substance, Epikuron 170 and the sorbic acid, and dissolve, with stirring. Filter the solution in sterile conditions through a nylon filter. The solution is clear, particle-free, and of a yellow color.

The taxoid active substance used is known from the literature as 5β, 20-epoxy-1, 2α, 4, 7β, 10β, 13α, 14β-heptahydroxytax-11-en-9-one 1,14-carbonate-4,10-diacetate-2-benzoate, 13-[(2R,3S)-3-(N-tert-butoxycarbonyl)-amino-2-hydroxy-5-methylhexanoate] from U.S. Pat. No. 5,705,508 (in which it is called SB-T-101131).

Examples for the production of formulations using the mixer:

### Example 7

### Production of a Formulation Without Any Active Substance

| | |
|---|---|
| Epikuron 170 | 1% |
| Ethanol | 2.7% |
| PEG 400 | 6.3% |
| Glucose | 5% |
| Water for injection | ad 100% |

Production: First the concentrate is produced according to example 1. For the diluting solution, the glucose is dissolved in the water. The two solutions are each drawn into a 50 ml Perfusor® syringe and each is inserted in a Perfusor® Compact syringe pump. The syringes are connected to a mixer according to FIG. 1 with a nozzle cross-section of 100 μm by hoses, and the inlets of the mixer are provided with non-return valves.

Then the concentrate is pumped into the mixer at a flow rate of 10 ml/h and the diluent at 90 ml/h.

With a radius of 500 μm for the discharge channel, a viscosity of 1.7 mPas and a density of 1.01 g/ml, a K value of 1036 is obtained according to Eq. 1. The mixer is operating in the turbulent range.

After a starting time of approx. 3 minutes, the mixture is collected.

The mixture is measured by photon correlation spectroscopy (Brookhaven BI 90) at 25° C. and 90° measuring angle. A mean hydrodynamic diameter of 106 nm is found, with a polydispersity index of 0.26.

### Example 8

### Production of a Nimodipine Formulation

| | |
|---|---|
| Nimodipine | 0.1% |
| Epikuron 170 | 1% |
| Ethanol | 2.7% |
| PEG 400 | 6.2% |
| Glucose | 5% |
| Water for injection | ad 100% |

Production is carried out as in Example 7.

The mixture is measured by photon correlation spectroscopy (Brookhaven BI 90) at 25° C. and 90° measuring angle. A mean hydrodynamic diameter of 119 nm is found, with a polydispersity index of 0.24.

### Example 9

### Production of an Ibuprofen Formulation

| | |
|---|---|
| Ibuprofen | 0.1% |
| Epikuron 170 | 1% |
| Ethanol | 2.7% |
| PEG 400 | 6.2% |
| Glucose | 5% |
| Water for injection | ad 100% |

Production is carried out as in Example 7.

The mixture is measured by photon correlation spectroscopy (Brookhaven BI 90) at 25° C. and 90° measuring angle. A mean hydrodynamic diameter of 103 nm is found, with a polydispersity index of 0.24.

### Example 10

### Production of a Clotrimazole Formulation

| | |
|---|---|
| Clotrimazole | 0.05% |
| Epikuron 170 | 1% |
| Ethanol | 2.7% |
| PEG 400 | 6.25% |
| Glucose | 5% |
| Water for injection | ad 100% |

Production is carried out as in Example 7.

The mixture is measured by photon correlation spectroscopy (Brookhaven BI 90) at 25° C. and 90° measuring angle. A mean hydrodynamic diameter of 95 nm is found, with a polydispersity index of 0.24.

### Example 11

### Production of a Paclitaxel Formulation

| | |
|---|---|
| Paclitaxel | 0.1% |
| Epikuron 170 | 1% |
| Ethanol | 2.7% |
| PEG 400 | 6.2% |
| Glucose | 5% |
| Water for injection | ad |
| | 100% |

Production is carried out as in Example 7.

The mixture is measured by photon correlation spectroscopy (Brookhaven BI 90) at 25° C. and 90° measuring angle. A mean hydrodynamic diameter of 114 nm is found, with a polydispersity index of 0.24.

### Example 12

### Production of a Formulation Of a Taxoid Active Substance

| | |
|---|---|
| Taxoid active substance | 0.1% |
| Epikuron 170 | 1% |
| Acid. Sorbic. | 0.05% |
| Ethanol | 2.7% |
| PEG 400 | 6.2% |
| Glucose | 5% |
| Water for injection | ad |
| | 100% |

Production is carried out as in Example 7.

The mixture is measured by photon correlation spectroscopy (Brookhaven BI 90) at 25° C. and 90° measuring angle. A mean hydrodynamic diameter of 156 nm is found, with a polydispersity index of 0.26.

The taxoid active substance used is known from the literature as 5β, 20-epoxy-1, 2α, 4, 7β, 10β, 13α, 14β-heptahydroxytax-11-en-9-one 1,14-carbonate-4,10-diacetate-2-benzoate, 13-[(2R,3S)-3-(N-tert-butoxycarbonyl)-amino-2-hydroxy-5-methylhexanoate] from U.S. Pat. No. 5,705,508 (in which it is called SB-T-101131).

### Example 13

### Production of Poloxamer-Stabilized Formulations

| | | |
|---|---|---|
| Concentrate 1: | | |
| Clotrimazole | 100 | mg |
| PEG 400 | ad 10 | g |
| Concentrate 2: | | |
| Ibuprofen | 100 | mg |
| PEG 400 | ad 10 | g |
| Continuous phase: | | |
| Poloxamer 408 | 1 | g |
| NaCl 0.9% | ad 100 | ml |

The two concentrates and the continuous phase were produced by dissolving the solid substances in the solvents, with stirring. The solutions were filtered through a 0.22 μm filter before use.

Production of the mixtures was carried out as in example 7, in each case using a mixing ratio between concentrate and continuous phase of 1+9. The overall flow rate was 100 ml/h.

The dispersity was determined by measuring the particle size in the photon correlation spectroscope.

A dispersity of 20-25 nm was found for the two mixtures of concentrates with the continuous phase. This dispersity could only be achieved when the poloxamer was added to the continuous phase.

In another study, the continuous phase was investigated on its own. Once again a particle size of 20-25 nm was found, and this can be attributed to the formation of poloxamer aggregates.

This means that in the present system the active substance is encapsulated by the poloxamer in the form of micelles and its precipitation is prevented.

1. A method for the production of nanodispersions, characterized in that at least two metered partial streams are brought together in such a way that they are subject to thorough mixing caused by turbulence, in which the partial streams have a flow rate in the range from 0.1 to 500 ml/h and the mixed stream has an overall flow rate in the range from 1 ml/h to 500 ml/h, preferably in the range from 10 to 200 ml/h, and during the turbulent mixing there is production of a disperse phase with a dispersity in the range from 0.1 to 5000 nm, preferably in the range from 10 to 1000 nm, and especially in the range from 10 to 200 nm.

2. A method according to claim 1, characterized in that the turbulent mixing arises because the partial streams flow through a nozzle into a discharge channel, the nozzle having a smaller diameter than the discharge channel.

3. A method according to claim 1 or 2, characterized in that the geometric parameters of the nozzle are chosen so as to obtain a characteristic number K of at least 250 according to the formula
$$K = \frac{{r}_{channel} \cdot ρ \cdot \dot{v}}{η \cdot {r}_{nozzle}^{2} \cdot π} ,$$
where r_{channel} denotes the radius of the discharge channel, ρ is the density of the mixture, \dot{v} is the overall flow rate, η is the viscosity of the mixture, r_{nozzle} is the radius of the nozzle and π is the ratio of the circumference of a circle to its diameter.

4. A method according to claim 2 or 3, characterized in that the discharge channel has a diameter between 0.2 and 2 mm and the nozzle has a diameter in the range from 10 to 500 μm.

5. A method according to one of the claims 2 to 4, characterized in that the length of the discharge channel is at least 10 times greater than the diameter of the discharge channel.

6. A method according to one of the claims 1 to 4, characterized in that the turbulent mixing is produced by a mixer, which comprises a first feed channel (13a) and a second feed channel (13b), which open into a nozzle (11), which is connected in turn to a following discharge channel (12), the discharge channel (12) being arranged in the extension of the first feed channel (13a) and at an angle in the range between 60° and 90°, preferably 90° to the second feed channel (13b).

7. A method according to one of the claims 1 to 6, characterized in that several mixers are connected in parallel or one after another.

8. A method according to one of the claims 1 to 7, characterized in that the mixed stream has a viscosity in the range from 0.7 to 150 mPas.

9. A method according to one of the claims 1 to 8, characterized in that the mixed stream has a density in the range from 700 kg/m³ to 1500 kg/m³.

10. A method according to one of the claims 1 to 9, characterized in that a first partial stream contains a substance or a mixture of substances which is sparingly soluble in a continuous phase, and a second partial stream contains the continuous phase or parts thereof and in that during turbulent mixing of the partial streams the disperse phase forms in a continuous phase.

11. A method according to one of the claims 1 to 10, characterized in that the disperse phase in its entirety or parts thereof consists of a therapeutically active substance, a pharmaceutical or some other active agent.

12. A method according to claim 11, characterized in that the substance is sparingly soluble in water, so that at least 100 parts water are required for dissolving 1 part of the substance.

13. A method according to claim 11 or 12, characterized in that the substance is a pharmaceutical from the following group: cardiovascular drugs, cancer drugs, virostatic agents, chemotherapeutic agents, hepatitis drugs, analgesics, antibiotics or immunomodulators.

14. A method according to one of the claims 1 to 13, characterized in that the disperse phase is formed either by precipitation on account of saturation of the solution, by a neutralization reaction, by an interaction between differently charged molecules, by association of molecules, by recomplexing or by a chemical reaction.

15. A method according to one of the claims 1 to 14, characterized in that the disperse phase is a solid or a mixture of different solids.

16. A method according to one of the claims 1 to 14, characterized in that the disperse phase is a liquid or a mixture of different liquids or a liquid-crystal phase.

17. A method according to one of the claims 1 to 14, characterized in that the disperse phase is formed mainly by phospholipid adducts from a first partial stream.

18. A method according to claim 17, characterized in that the phospholipid is a phospholipid, a hydrogenated or partially hydrogenated phospholipid, a lysophospholipid or a ceramide, preferably one of the phospholipids with the trivial names lecithin or kephalin, quite especially preferred a purified lecithin from soybeans of the grades Epikuron 170, Epikuron 175, Lipoid S100 or S75 or a purified lecithin from egg yolk of the grades Lipoid E80, E100 and EPC or mixtures of these compounds, and the proportion by weight of the phospholipid in the first partial stream is between 0.01% and 40%, preferably between 5% and 20%, and especially between 9 and 11%.

19. A method according to one of the claims 17 to 18, characterized in that a first partial stream is a solution of a phospholipid or the mixture of phospholipids in an organic, water-miscible, preferably anhydrous solvent.

20. A method according to claim 19, characterized in that the solvent contains 10 to 50, preferably 25 to 35 parts by weight ethanol and 50 to 90, preferably 65 to 75 parts by weight polyethyleneglycol 400 (PEG 400).

21. A method according to claim 19 or 20, characterized in that other substances for increasing the shelf life, chemical and physical stability, and for regulating the pH or the viscosity, are added to the solvent.

22. A method according to one of the claims 1 to 14, characterized in that the disperse phase or parts of the disperse phase is a gas or a mixture of gases.

23. A method according to one of the claims 1 to 22, characterized in that the continuous phase is water or distilled water or an aqueous medium or an aqueous medium with additions of electrolytes, monosaccharides or disaccharides, alcohols, polyols or their mixtures.

24. A method according to claim 23, characterized in that the continuous phase contains one or more viscosity-raising substances.

25. A method according to one of the claims 23 or 24, characterized in that the continuous phase contains stabilizers and/or surfactants.

26. A method according to one of the claims 23 to 25, characterized in that the continuous phase contains block copolymers from the poloxamer group.

27. A method according to one of the claims 23 to 26, characterized in that substances are added to the continuous phase for the purpose of making it isotonic or euhydric, for increasing the physical and/or chemical stability and shelf life and for preventing microbiological spoilage.

28. A method according to one of the claims 1 to 27, characterized in that at least one of the partial streams contains an organic solvent, preferably polyethyleneglycol, propyleneglycol, ethanol, glycofurol, glycerol or other organic solvents or mixtures thereof that are suitable for application in humans or animals.

29. A method for the in-situ formulation of a pharmaceutical dispersion according to one of the claims 1 to 28, in which the quantity of pharmaceutical dispersion produced in unit time corresponds to the quantity to be applied.

30. A method for the in-situ formulation of a pharmaceutical dispersion according to claim 29, characterized in that the pharmaceutical dispersion is applied parenterally to humans or animals.

31. A method for the in-situ formulation of a pharmaceutical dispersion according to claim 29 or 30, characterized in that the pharmaceutical dispersion is applied to humans or animals by the oral, ophthalmologic, otologic, topical, nasal, vaginal, urethral or rectal route.
